(19) United States
(12) Patent Application Publication
(10) Pub. No.: US 2003/0032931 A1
(43) Pub. Date: Feb. 13, 2003

(54) Device for draining uncontrolled urine release in male persons

(76) Inventors: Reinhold Grundke, Burghausen (DE); Bernhard Liedl, Munich (DE); Wilhelm Jaeniche, Kehl-Leutesheim (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/216,381

(22) Filed: Aug. 12, 2002

(30) Foreign Application Priority Data
Aug. 13, 2001 (DE) 201 13 445.4

(51) Int. Cl. A61F 5/44

(52) U.S. Cl. 604/349; 604/346; 604/347

(57) ABSTRACT

A device for draining uncontrolled urine release in male persons has an integrally formed main body of silicone rubber. The main body comprises in a middle area, a cylindrical chamber portion having an internal cross section which is greater than the cross section of a penis of an user, in a front area, a funnel portion adjoining said cylindrical chamber portion, tapering toward the front of main said body and merging into a hose connection, and in a rear area, a transition portion adjoing said chamber portion, tapers toward the rear of said body and merging into a cylindrical terminal portion having an internal diameter which is slightly smaller than the external diameter of said penis of said user, at least two expansion tongues being provided on the rear end of said terminal portion.

The invention relates to a device for draining uncontrolled urine release in male persons, with a main body which has a chamber portion adjoined, in the front area, by a funnel portion which tapers toward the front and which merges into a hose connection, the rear area of the main body being provided with means for securing the device to the penis of a user.

EP 0 824 905 A1 discloses a device for draining uncontrolled urine release in male persons, which device has a main body made of soft plastic. The middle portion of the main body forms a cylindrical jacket which is adapted to the penis cross section and which is adjoined at one end by a funnel chamber which narrows in a funnel shape in the direction of a hose connection. At the other end the jacket is subdivided into a plurality of tongues by interruptions succeeding one another in the circumferential direction. Moreover, a soft elastic pliable sleeve can be attached to the main body at the transition from the closed jacket to the tongues, which soft elastic pliable sleeve is suitable for surrounding the tongues elastically from the outside. Provided on the jacket there is an outwardly extending connection point for a rinsing device.

Especially in elderly persons with poor motor response, it has been found that applying the known device is too complicated because it is in two parts, with the result that the device has often been poorly fitted. In addition, it can happen that urine can escape through the interruptions between two adjacent tongues.

The object of the invention is to make available, by structurally simple means, a device for draining uncontrolled urine release in male persons, which device can be applied easily and guarantees that no urine can escape at the rear end.

This object is achieved by means of a device for draining uncontrolled urine release in male persons, the device having an integrally formed main body of silicone rubber, the main body comprising:

in a middle area, a cylindrical chamber portion having an internal cross section which is greater than the cross section of a penis of an user,

in a front area, a funnel portion adjoining said cylindrical chamber portion, tapering toward the front of main said body and merging into a hose connection, and

in a rear area, a transition portion adjoing said chamber portion, tapers toward the rear of said body and merging into a cylindrical terminal portion having an internal diameter which is slightly smaller than the external diameter of said penis of said user, at least two expansion tongues being provided on the rear end of said terminal portion.

The device according to the invention can be applied easily to the penis of a user. To do so, an expansion tongue is in each case gripped with one hand and the terminal portion is expanded until the penis can be inserted into the main body via the terminal portion. After insertion, the expansion tongues are released, as a result of which the terminal portion closes snugly about the shaft of the penis in a manner leaktight to urine. In the area of the terminal portion, the silicone rubber material is configured in such a way that no urine can escape but at the same time no blood congestion is caused. Since the device is made of silicone rubber, it is easy to clean and does not trigger any allergies.

In a prefered embodiment of the device of the present invention in the area of said transition portion a tubular rinsing and ventilation connection is provided, said tubular rinsing and ventilation connection having an outer, free end face lying within the imaginary continuation of the outer surface of said chamber portion. Chafing by the rinsing and ventilation attachment is prevented in this way.

Preferably two expansion tongues are provided diametrically opposite to each other at the rear end of said terminal portion.

In addition, at least one securing tongue can be provided on the rear end of said terminal portion and can be used to secure the device to underwear or to a belt.

An illustrative embodiment of the invention is explained in more detail below with reference to drawings, in which:

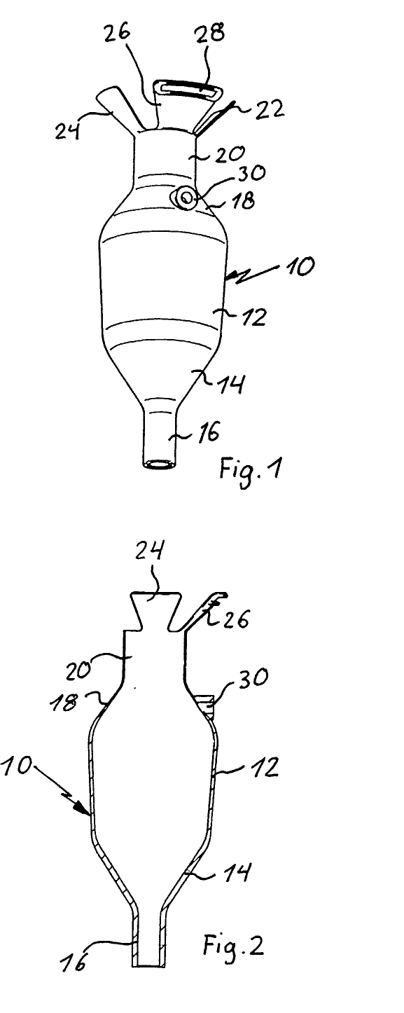

FIG. 1 shows a perspective view of a device for draining uncontrolled urine release in male persons;

FIG. 2 shows a longitudinal section through the device from FIG. 1.

The device for draining uncontrolled urine release in male persons has a integrally formed, rotationally symmetrical main body 10 made of silicone rubber. In its middle area, the main body 10 comprises a cylindrical chamber portion 12 whose internal diameter is greater than the external diameter of the penis of a user. Adjoining this chamber portion 12 at its front (toward the bottom in FIG. 1) there is a funnel portion 14 which tapers toward the front and which at its front end merges into a hose connection 16. On the side of the chamber portion 12 remote from the funnel portion 14, the chamber portion 12 merges into a transition portion 18 which has the shape of a truncated cone and which tapers conically toward the rear (toward the top in FIG. 1). Adjoining this transition portion 18 there is a cylindrical terminal portion 20 whose internal diameter is slightly smaller than the external diameter of the penis of a user. The expression “slightly smaller” is to be understood as meaning, for example, that the internal diameter is approximately 20 mm for a penis diameter of 22 to 40 mm. Two expansion tongues 22, 24 lying diametrically opposite one another are provided on the rear end of the terminal portion 20. Arranged between the expansion tongues 22, 24 there is a securing tongue 26 which has an eyelet 28 adjacent to its free end.

Provided in the area of the transition portion 18 there is a tubular rinsing and ventilation attachment 30 which extends radially with respect to the center axis of the main body 10 and communicates with the interior of the main body 10. As can be seen from FIG. 2, the radially outer end face of the rinsing and ventilation attachment 30 lies within the imaginary continuation of the outer cylindrical surface of the chamber portion 12.

To correctly apply the device according to the invention on the penis of a user, the expansion tongues 22, 24 are gripped and are pulled outward radially with respect to the center axis of the main body 10, by which means the internal cross section of the terminal portion 20 is enlarged to such an extent that the penis can be inserted into the main body 10. The expansion tongues 22, 24 are then released, by which means the terminal portion 20 closes snugly onto the penis with a press force which prevents escape of urine via the terminal portion 20. At the same time, the press force is configured such that there is no congestion of blood in the penis.

The device is then secured to a belt or the like via the eyelet 28 of the securing tongue 26. Instead of the eyelet 28, a velcro tape or the like can also be arranged on the securing tongue 26.

The dimensions of the device according to the invention can be configured in accordance with the size of a user's penis. In a device configured for the average user, the internal diameter of the chamber portion is approximately 43 mm. The terminal portion 20 in this embodiment preferably has an internal diameter of 20 mm and an external diameter of 21 mm. The length of the terminal portion 20 is approx. 17.5 mm. Such an embodiment is suitable for a penis diameter of between 22 and 40 mm.

1. A device for draining uncontrolled urine release in male persons, the device having an integrally formed main body of silicone rubber, the main body comprising:
in a middle area, a cylindrical chamber portion having an internal cross section which is greater than the cross section of a penis of an user,
in a front area, a funnel portion adjoining said cylindrical chamber portion, tapering toward the front of main said body and merging into a hose connection, and
in a rear area, a transition portion adjoing said chamber portion, tapers toward the rear of said body and merging into a cylindrical terminal portion having an internal diameter which is slightly smaller than the external diameter of said penis of said user, at least two expansion tongues being provided on the rear end of said terminal portion.

2. The device as claimed in claim 1, wherein, in the area of said transition portion a tubular rinsing and ventilation connection is provided, said tubular rinsing and ventilation connection having an outer, free end face lying within the imaginary continuation of the outer surface of said chamber portion.

3. The device as claimed in claim 1, wherein two expansion tongues are provided diametrically opposite to each other at the rear end of said terminal portion.

4. The device as claimed in claim 1, wherein at least one securing tongue is provided on the rear end of said terminal portion.
